(19) United States
(12) Patent Application Publication
(10) Pub. No.: US 2025/0036834 A1
(43) Pub. Date: Jan. 30, 2025

(54) Chemical Simulation Apparatus and Method

(71) Applicants: HYUNDAI MOTOR COMPANY, SEOUL (KR); KIA CORPORATION, SEOUL (KR)

(72) Inventors: Kyujin SHIN, Gunpo-si (KR); Seunghyo NOH, Uiwang-si (KR)

(21) Appl. No.: 18/504,749

(22) Filed: Nov. 8, 2023

(30) Foreign Application Priority Data
Jul. 24, 2023 (KR) 10-2023-0096186

(51) Int. Cl. G06F 30/27

(52) U.S. Cl. CPC G06F 30/27

(57) ABSTRACT

A chemical simulation apparatus may include a training data generation module configured to sample a molecular structure to be learned, set a wave function, and extract an energy value with respect to the molecular structure based on a quantum computing, a fingerprint conversion module configured to convert structure calculated in the quantum computing into a fingerprint, a learning module configured to perform a neural network learning by using the converted fingerprint as an input, and a prediction module configured to predict material energy or physical properties by using the learned neural network.

### CROSS REFERENCE TO RELATED APPLICATIONS

This application claims priority to and the benefit of Korean Patent Application No. 10-2023-0096186 filed in the Korean Intellectual Property Office on Jul. 24, 2023, the entire contents of which is incorporated herein by reference.

### BACKGROUND

### (a) Field

The present disclosure relates to a chemical simulation apparatus and method. Specifically, the present disclosure relates to a chemical simulation apparatus and method capable of extending simulation model through machine learning.

### (b) Description of the Related Art

Quantum computing refers to a computing method that processes information using quantum mechanical principles. Unlike classical computers that process data using bits, quantum computers use quantum-bits or qubits. Quantum superposition and quantum entanglement are representative characteristics of qubits. Quantum superposition means that a quantum state is represented as a linear sum of all possibilities, and whereas classical bits may only have one state (0 or 1) at a time, qubits may have superposed states. That is, a qubit may have states 0 and 1 at the same time. As the number of qubits increases, the number of possible states that the computer may simultaneously consider also increases exponentially. On the other hand, quantum entanglement refers to a phenomenon in which two qubits are connected to each other and the state of one qubit directly affects the state of the other qubit. Entangled qubits may immediately perform complex interactions, and certain types of calculations may be performed very efficiently by using this.

The quantum classical hybrid algorithm is an algorithm that finds an optimal solution by exchanging data between a quantum computer and a classical computer, and may aim to find a minimum eigenvalue of the Hamiltonian. Regarding quantum chemical operations, a quantum computer may simulate the quantum state of molecules, represent the quantum state of qubit ansatz determined by parameters, and measure the Hamiltonian thereof, and a classical computer may calculate an energy from the measured Hamiltonian value and determine a new parameter that minimizes this energy. Using this, a new quantum state may be prepared and optimized in a subsequent step of quantum operation. However, due to the slow hardware development speed of quantum computers, the number of molecular quanta capable of such calculation is limited and practicality is not high. Accordingly, a plan to expand the simulation scale beyond the current hardware limitations is required.

The above information disclosed in this Background section is only for enhancement of understanding of the background of the disclosure, and therefore it may contain information that does not form the prior art that is already known in this country to a person of ordinary skill in the art.

### SUMMARY

The present disclosure attempts to provide a chemical simulation apparatus and method capable of substantially expand a chemical simulation scale by converging quantum computing technology and artificial intelligence technology.

Also, the present disclosure attempts to provide a chemical simulation apparatus and method capable of predicting physical properties and energy of the material structure expanded based on the sampled molecular structure by using the energy of the sampled molecular structure obtained through quantum classical algorithm as an input value of an artificial neural network for prediction of molecule physical properties.

A chemical simulation apparatus may include a training data generation module configured to sample a molecular structure to be learned, set a wave function, and extract an energy value with respect to the molecular structure based on a quantum computing, a fingerprint conversion module configured to convert structure calculated in the quantum computing into a fingerprint, a learning module configured to perform a neural network learning by using the converted fingerprint as an input, and a prediction module configured to predict material energy or physical properties by using the learned neural network.

The training data generation module may be configured to set an ansatz including a quantum circuit configured to implement a quantum algorithm taking the sampled molecular structure as an input.

The training data generation module may be configured to perform the quantum algorithm based on the set wave function, and optimize a ground state energy with respect to the molecular structure.

The training data generation module may be configured to extract the energy value with respect to all molecular structures determined to be necessary for the neural network learning.

The fingerprint conversion module may be configured to obtain the fingerprint by applying a symmetric function with respect to the molecular structure, where the fingerprint is generated as a fingerprint of a same structure with respect to a same molecular structure, regardless of translation and rotation.

The symmetric function may include a symmetric function of Gaussian type.

The learning module may be configured to finish learning when a calculation error of a training set converges to a predetermined value or less.

The predetermined value may be 10 mHa/atom, where mHa denotes milli-Hartree.

The learning module may be configured to finish learning when a prediction accuracy of a test set not used for the learning converges to the predetermined value, modify the training set when the prediction accuracy does not converge to the predetermined value, the fingerprint conversion module may be configured to convert the fingerprint with respect to the modified training set, and the learning module may be configured to perform the neural network learning by using the fingerprint converted with respect to the modified training set as an input.

The prediction module may be configured to predict new structure not included in the neural network learning.

A chemical simulation method may include sampling a molecular structure to be learned, setting a wave function, extracting an energy value with respect to the molecular structure based on a quantum computing, converting a structure calculated in the quantum computing into a fingerprint, performing a neural network learning by using the converted fingerprint as an input, and predicting a material energy or physical properties by using the learned neural network.

A chemical simulation method may further include setting an ansatz including a quantum circuit configured to implement a quantum algorithm taking the sampled molecular structure as an input.

A chemical simulation method may further include performing the quantum algorithm based on the set wave function, and optimizing a ground state energy with respect to the molecular structure.

The extracting the energy value may include extracting the energy value with respect to all molecular structures determined to be necessary for the neural network learning.

The converting into the fingerprint may include obtaining the fingerprint by applying a symmetric function with respect to the molecular structure, where the fingerprint is generated as a fingerprint of a same structure with respect to a same molecular structure, regardless of translation and rotation.

The symmetric function may include a symmetric function of Gaussian type.

The performing of the neural network learning may include finishing learning when a calculation error of a training set converges to a predetermined value or less.

The performing of the neural network learning may include finishing learning when prediction accuracy of a test set not used for the learning converges to the predetermined value, and modifying the training set when the prediction accuracy does not converge to the predetermined value, the converting into the fingerprint may include converting the fingerprint with respect to the modified training set, and the performing the neural network learning may include performing the neural network learning by using the fingerprint converted with respect to the modified training set as an input.

A computer-readable medium may store a program or instructions to enable a computer to execute a method, where the computer may include a processor configured to execute the program or instructions, where the method may include sampling a molecular structure to be learned, setting a wave function, extracting an energy value with respect to the molecular structure based on a quantum computing, converting a structure calculated in the quantum computing into a fingerprint, performing a neural network learning by using the converted fingerprint as an input, and predicting a material energy or physical properties by using the learned neural network.

Unlike a chemical simulation scale expansion method of the quantum computing-based quantum classical algorithm such as the conventional VQE algorithm, physical properties and energy of a material and a molecular structure of which the scope of objects is expanded may be predicted by inputting energy of an object molecular structure calculated by the quantum classical algorithm as training data of a machine learning-based artificial neural network. Through the linkage of the quantum computing-based simulation with machine learning, a computational scale of an existing chemical simulation quantum classical algorithm may be increased by more than about 100 times. In addition, embodiments may be modified and optimized through appropriate selection of quantum simulation method, quantum classical algorithm, machine learning model, or the like depending on molecular structures to be simulated.

Particularly, since the scale of the chemical simulation is small (e.g., 10 to 20 qubit level) due to the limit of existing quantum computer hardware, it may be difficult to apply the quantum calculation to a computational chemistry problem of a practical scale. In addition, since a computational scale of DMET, FMO, or the like may not be expanded to a sufficiently large scale to have practicality, it is still difficult in the current situation to solve a chemistry problem in a practical scale. However, according to the present embodiment, the chemical simulation may be expanded to a sufficiently large scale by using a quantum computing result as of a learning input value a machine learning model, and linking a conventional quantum computing algorithm of a small scale with machine learning. In addition, since the quantum computing algorithm and the machine learning algorithm may be tuned in a direction desired by the user, each algorithm may be configured to become an optimal combination, and the quantum classical algorithm and the machine learning algorithm selected as such may complement calculation accuracy and calculation speed and scale of each other.

### BRIEF DESCRIPTION OF THE FIGURES

FIG. 1 is a drawing for explaining a chemical simulation apparatus according to an embodiment.

FIG. 2 is a drawing for explaining a chemical simulation method according to an embodiment.

FIG. 3 is a drawing for explaining a training data generation module according to an embodiment.

FIG. 4 is a drawing for explaining a fingerprint conversion module according to an embodiment.

FIG. 5 is a drawing for explaining a learning module according to an embodiment.

FIG. 6 is a drawing for explaining a prediction module according to an embodiment.

FIG. 7 is a drawing for explaining a computing device according to an embodiment.

### DETAILED DESCRIPTION

The present disclosure will be described more fully hereinafter with reference to the accompanying drawings, in which embodiments of the disclosure are shown. As those skilled in the art would realize, the described embodiments may be modified in various different ways, all without departing from the spirit or scope of the present disclosure. Accordingly, the drawings and description are to be regarded as illustrative in nature and not restrictive. Like reference numerals designate like elements throughout the specification.

Throughout the specification and claims, when a part “includes” a certain element, it means that other elements may be further included, rather than excluding other elements, unless otherwise stated. Terms including ordinal numbers such as first, second, and the like will be used only to describe various constituent elements, and are not to be interpreted as limiting these constituent elements. The terms are only used to differentiate one constituent element from other constituent elements.

In addition, terms such as “ . . . part,” “ . . . portion,” “ . . . er/or,” or “module” disclosed in the present specification may mean a unit that may process at least one function or operation described in this specification, and this may be implemented by hardware, software, or a combination thereof.

A quantum classical hybrid algorithm may use a variational quantum eigensolver (VQE) for quantum chemical simulations, including calculation of a ground state energy of a molecule. The VQE is one of algorithms of a quantum classical hybrid computing method that utilizes a classical computer together to compensate for the performance of an incomplete quantum computer, and may be mainly used to estimate the energy of the ground state (lowest energy state). The VQU may be particularly useful in quantum chemistry, and may be used to calculate the ground state energy (i.e., an intrinsic energy that a substance has in its most stable state, e.g., 0° K) of a molecule.

A noisy intermediate-scale quantum (NISQ) device means a quantum computer that allows a certain level of error (for example, an error generated from qubit interaction), and may mean a hardware that may be practically utilized in the current stage, which is before a quantum computer composed of qubits capable of perfectly controlling the quantum state is utilized. Although the NISQ device may not execute a very complicated quantum algorithm without being fully implemented with such quantum error modification, it shows an excellent performance better than a classical computer, on some works such as a quantum chemistry problem for calculating an energy of a specific molecule, or a certain optimization problem.

In order to increase the computational scale of the existing NISQ device, there are attempts to modify input values required to the VQE algorithm by method such as a density matrix embedding theory (DMET), fragment molecular orbital (FMO), or the like. The DMET is one of the methodologies for describing complex quantum systems, in which the whole system may be divided into smaller subsystems (or “embeddings”), each of which may be treated. This approach may reduce the computational complexity of the overall system. On the other hand, the FMO is another computational chemistry methodology that may be used to analyze large-scale molecular systems. The FMO may separate the entire molecule into several fragments, calculate the electronic structure of each fragment independently, and then combine them to obtain the electronic structure of the entire system.

However, these attempts have not reached the stage where practical predictions such as prediction of material properties are possible, due to hardware limitations of quantum computers. For example, simulations of at least 1,000 molecules are required to be used for predicting material properties, but current attempts fall far short of this.

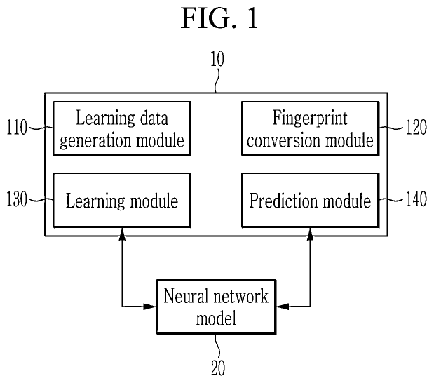

FIG. 1 is a drawing for explaining a chemical simulation apparatus according to an embodiment.

Referring to FIG. 1, a chemical simulation apparatus 10 according to an embodiment may significantly expand the quantum computing scale by using the result obtained by the VQE algorithm as an input value of an artificial neural network (or neural network). For example, a neural network may predict multi-molecular energy by learning molecular structure energy calculated by a quantum algorithm. Accordingly, it is possible to improve the versatility of material design and the accuracy of predicting physical properties, and by utilizing the extensibility of the neural network model, the quantum chemistry simulation scale of the NISQ device, such as molecular size and number of molecules, may be greatly increased. To this end, a chemical simulation apparatus 10 may include a training data generation module 110, a fingerprint conversion module 120, a learning module 130, and a prediction module 140.

The training data generation module 110 may generate quantum computer-based training data. Specifically, the training data generation module 110 may sample a molecular structure to be learned, set a wave function, and extract an energy value with respect to the molecular structure based on the quantum computing. Here, the wave function may be used to effectively input the sampled molecular structure to the quantum algorithm. That is, the training data generation module 110 may set an ansatz including a quantum circuit configured to implement the quantum algorithm taking the sampled molecular structure as an input.

The ansatsu is used to set up initial estimation values or hypotheses that have not yet been proven in order to solve a problem or an equation. In quantum computing, ansatsu may be used to denote a formula or a function that is primarily used to describe quantum states. It may be a mathematical expression representing an array of qubits, which is generally used to represent a final state of a certain system. In the VQE algorithm, it is assumed that ansatsu represents a specific quantum state, and through this, parameters may be adjusted to obtain optimal results. Here, the ansatsu determines the structure of the quantum circuit, which may be used to prepare an initial state, apply a quantum operation, and measure the result.

The training data generation module 110 may perform the quantum algorithm based on a set wave function, and optimize the ground state energy with respect to the molecular structure. In addition, the training data generation module 110 may extract energy value with respect to all molecular structures determined to be necessary for a neural network learning. Specifically, the training data generation module 110 may perform quantum calculation at a theoretical level through quantum computing and extract an energy value for a required molecular structure. These operations may be performed such that the calculation error may be the predetermined value or less, and in some embodiments, the predetermined value may be 10 mHa/atom, where mHa denotes milli-Hartree.

In some embodiments, theoretical solution may be a result of full configuration interaction (FCI) calculation. As one of the calculation methodologies used in the quantum chemistry, the FCI is used to accurately express the interactions of electrons, and as one of the calculation methodologies used in quantum chemistry, FCI is used to accurately express the interaction of electrons, and as an interpretation method that considers all interactions of electrons in a material, it may be a methodology that may calculate highly accurate energy. In particular, the FCI is one of the post-Hartree-Fock methods and may have a form of a linear combination of all possible excitation operators. However, for general materials, the calculation amount may increase very rapidly.

The fingerprint conversion module 120 may convert structure coordinates for artificial intelligence learning. Specifically, the fingerprint conversion module 120 may convert structure calculated in the quantum computing into fingerprint, and here, the structure calculated in the quantum computing may include a molecular structure image calculated by using a quantum computer.

The fingerprint conversion module 120 may obtain the fingerprint by applying a symmetric function with respect to a molecular structure (for example, 3-dimension structure). Here, regardless of translation and rotation, the fingerprint may be generated as a fingerprint of a same structure with respect to a same molecular structure, and even if the molecular structure is translated and rotated, the input value as the same structure may be provided to the neural network. In some embodiments, symmetric function may include a symmetric function of Gaussian type. The symmetric function of Gaussian type may mathematically express interaction between atoms by using a distance, an angle, or the like in a 3-dimensional space, and from this, characteristics of molecules according to a change of atomic distance may be expressed more accurately.

The learning module 130 may perform the neural network learning, that is, a learning with respect to a neural network model 20 by using the fingerprint converted by the fingerprint conversion module 120 as an input. The learning module 130 may perform learning while reducing an error between the energy of the training data calculated through a quantum computer and a result obtained through an arbitrary artificial neural network. Specifically, the learning module 130 may finish learning when a calculation error of a training set converges to the predetermined value or less, where the predetermined value may be 10 mHa/atom.

The learning module 130 may finish learning when prediction accuracy of a test set not used for the learning converges to the predetermined value, and modify the training set when the prediction accuracy does not converge to the predetermined value. When the training set is modified, the fingerprint conversion module 120 may convert the fingerprint with respect to the modified training set, and the learning module 130 may repeat performing the neural network learning by using the fingerprint converted with respect to the modified training set as an input.

The prediction module 140 may, by the learning module 130, predict material energy or physical properties by using the learned neural network, that is, the neural network model 20. In addition, the prediction module 140 may predict new structure not included in the neural network learning.

According to the present embodiment, unlike a chemical simulation scale expansion method of the quantum computing-based quantum classical algorithm such as the conventional the VQE algorithm, physical properties and energy of a material and a molecular structure of which the scope of objects is expanded may be predicted by inputting energy of an object molecular structure calculated by the quantum classical algorithm as the training data of a machine learning-based artificial neural network. Through the linkage of the quantum computing-based simulation with machine learning, a computational scale of the existing chemical simulation quantum classical algorithm may be increased by more than about 100 times. In addition, embodiments may be modified and optimized through appropriate selection of quantum simulation method, quantum classical algorithm, machine learning model, or the like depending on molecular structures to be simulated.

Particularly, since the scale of the chemical simulation is small (e.g., 10 to 20 qubit level) due to the limit of existing quantum computer hardware, it may be difficult to apply the quantum calculation to a computational chemistry problem of a practical scale. In addition, since a computational scale of the DMET, the FMO, or the like may not be expanded to a sufficiently large scale to have practicality, it is still difficult in the current situation to solve a chemistry problem in a practical scale. However, according to the present embodiment, the chemical simulation may be expanded to a sufficiently large scale by using a quantum computing result as of a learning input value a machine learning model, and linking a conventional quantum computing algorithm of a small scale with machine learning. In addition, since the quantum computing algorithm and the machine learning algorithm may be tuned in a direction desired by the user, each algorithm may be configured to become an optimal combination, and the quantum classical algorithm and the machine learning algorithm selected as such may complement calculation accuracy and calculation speed and scale of each other.

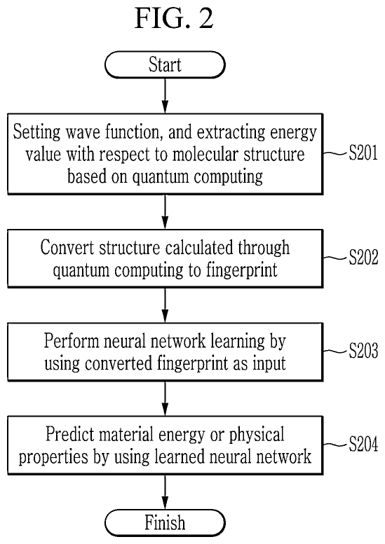

FIG. 2 is a drawing for explaining a chemical simulation method according to an embodiment.

Referring to FIG. 2, a chemical simulation method according to an embodiment may include a step S201 of sampling the molecular structure to be learned, setting the wave function, and extracting energy value with respect to the molecular structure based on the quantum computing, a step S202 of converting the structure calculated in the quantum computing into the fingerprint, a step S203 of performing the neural network learning by using the converted fingerprint as an input, and a step S204 of predicting a material energy or physical properties by using the learned neural network.

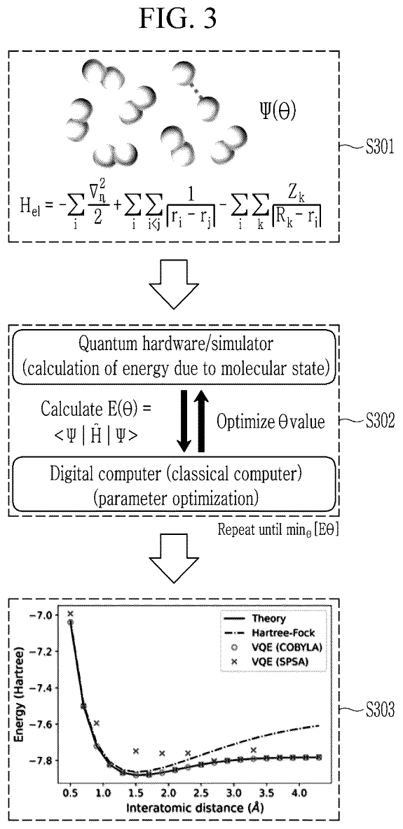

For a more detailed description of the method, reference may be made to the description described above with respect to FIG. 1 and the description to be described later with respect to FIG. 3 to FIG. 7, and thus duplicate descriptions will be omitted herein.

FIG. 3 is a drawing for explaining the training data generation module according to an embodiment.

Referring to FIG. 3, as shown in step S301, the training data generation module according to an embodiment 110 may sample the molecular structure, and configure the input value of the quantum algorithm. The training data generation module 110 may set the structure of a learning sample on which quantum calculation may be performed with a quantum computer that may be operated according to a specific implementation environment, where the structure of the learning sample may include a size, a shape, or the like of the molecular structure. In addition, the training data generation module 110 may select an input value appropriate for the molecular structure input to the quantum algorithm.

In addition, as shown in step S302, the training data generation module 110 may calculate the energy of the molecular structure based on the quantum computing. By using a quantum algorithm that may be implemented with a quantum processing unit (QPU) according to a specific implementation environment, including the VQE, it is possible to calculate the energy value of the input molecular structure and perform parameter optimization. At this time, calculation results corresponding to a range of predetermined reference errors may be collected.

In addition, as shown in step S303, the training data generation module 110 may continuously perform the quantum computing-based quantum calculation with respect to all the sampled molecular structures, and may extract continuous energy data (Potential Energy Surface, PES).

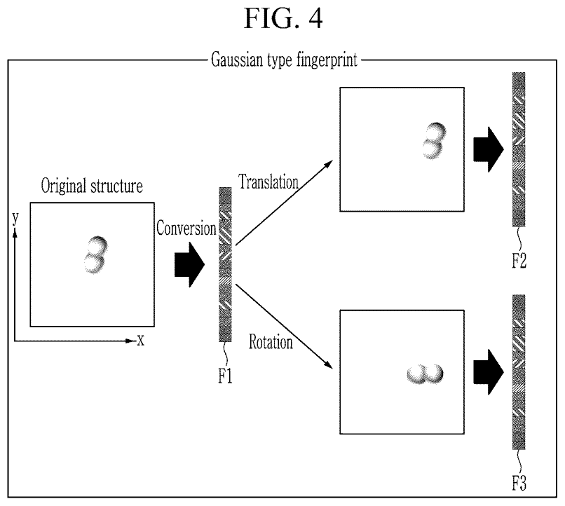

FIG. 4 is a drawing for explaining a fingerprint conversion module according to an embodiment.

Referring to FIG. 4, a fingerprint conversion module according to an embodiment 120 may convert 3-dimensional structure information into fingerprint. In order to learn the 3-dimensional molecular structure, it is necessary to provide the same input even if the molecular structure rotates and translates. When the conventional Cartesian Coordinates consisting of the actual position (x, y, z) are directly used, a different input value may be provided according to the translation and rotation of the molecular structure even if they have actually the same structure. Therefore, even if the molecular structure is rotated, it is necessary to output the same vector based on symmetry.

The fingerprint conversion module 120 may, by using the symmetric function of Gaussian type, convert a local environment mimicking a surrounding structure of the atom to a unique mathematical fingerprint, so as to recognize the same structure at any position as a unique number, and such generated input value may be used as the input value of the artificial neural network. The following represents the symmetric function of Gaussian type that may be used by the fingerprint conversion module 120.

$${G}_{i}^{II} = {\sum }_{j \neq i}^{\begin{matrix}atoms j within {R}_{c} \\ distance of atom i\end{matrix}} {e}^{\frac{- {η ( {R}_{ij} - {R}_{s} )}^{2}}{{R}_{c}^{2}}} {f}_{c} ( {R}_{ij} )$$

$${G}_{i}^{IV} = {2}^{1 - ζ} {\sum }_{\begin{matrix}j , k \neq i \\ ( j \neq k )\end{matrix}}^{\begin{matrix}atoms j within {R}_{c} \\ distance of atom i\end{matrix}} {( 1 + λ cos {θ}_{ijk} )}^{ζ} {e}^{\frac{- η ( {R}_{ij}^{2} + {R}_{ik}^{2} + {R}_{jk}^{2} )}{{R}_{c}^{2}}} {f}_{c} ( {R}_{ij} ) {f}_{c} ( {R}_{ik} ) {f}_{c} ( {R}_{jk} )$$

The symmetric function of the Gaussian type may include, for example, a function G2 for generating a fingerprint with respect to a distance between atoms and a function G4 for generating a fingerprint using an angle between atoms. Specifically, the function G2 and the function G4 are calculated for the local environment around a central atom, respectively, and the combined result may be used as a fingerprint. Therefore, the same vector values are output to be used as a fingerprint, only when the surrounding environment of the central atom is locally the same.

Accordingly, a fingerprint F1 generated from the original structure, a fingerprint F2 generated from a translated state of the original structure, and a fingerprint F3 generated from a rotated state of the original structure may be formed the same. Since the local environment of the central atom based on coordinates is not generated as the fingerprint but a unique vector value is generated as the fingerprint through distances and angles between the central atom and surrounding atoms, the same fingerprint as the original structure is generated when the surrounding environment of the central atom is not changed, even if the original structure is translated or rotated. That is, if the local environment around the central atom (for example, arrangement of atoms) is the same, then the same fingerprint value is output. In some embodiments, at this time, although the uniqueness of the fingerprint may be improved by setting various values of eta, gamma, and zeta, considering that in this case the calculation amount is simultaneously increased, eta, gamma, and zeta values may be set such that the fingerprint may be in an appropriate size.

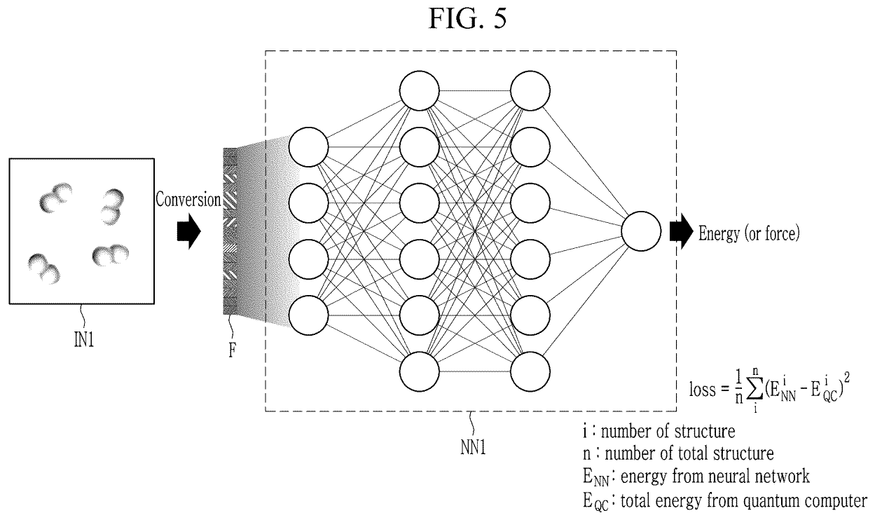

FIG. 5 is a drawing for explaining a learning module according to an embodiment.

Referring to FIG. 5, a learning module according to an embodiment 130 may enable the artificial neural network to learn the structure and energy of the quantum computer. The eigenvector F of the structure IN1 calculated above is provided as an input value to the artificial neural network NN1, and the total energy value of the entire structure may be used as an output. The learning module 130 may enable the artificial neural network to learn the energy of the quantum computer, by adjusting artificial neural network parameter (e.g., weight and bias) by error of the output value due to a structure input by using a back-propagation algorithm such that the error between the energy calculated by the quantum computer and the prediction result of the artificial neural network converges to 0. Particularly, the learning module 130 may, after predicting the energy with respect to the test set by using the artificial neural network, when an error of energy above 10 mHa/atom occurs, reconstruct the training set until the error decreases.

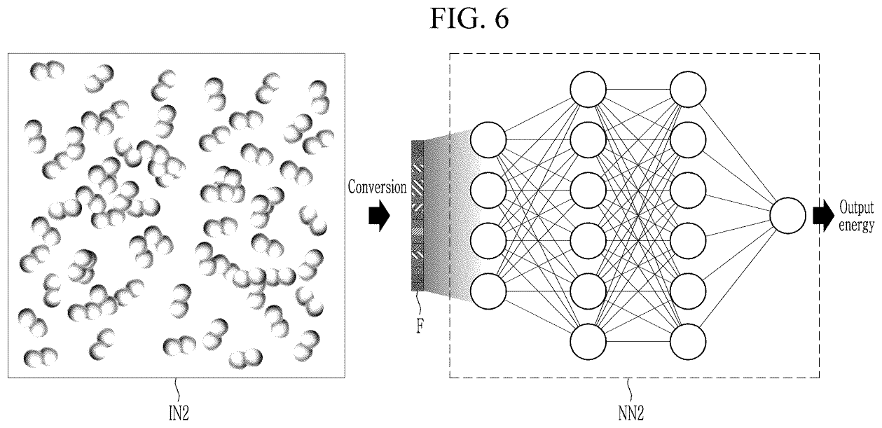

FIG. 6 is a drawing for explaining a prediction module according to an embodiment.

Referring to FIG. 6, the prediction module 140 according to an embodiment may predict the new structure through the trained artificial neural network NN2. The prediction module 140 may input the eigenvector F converted from the new structure IN2 into the artificial neural network NN2, and predict the new structure not included in the learning, or predict a molecular structure of a significantly larger scale than the scale of learning (for example, by more than 200 times), and may calculate physical properties of the material such as bonding strength, density, diffusion, by using the output energy value. Such prediction may be used not only for hydrogen molecules but also for all kinds of materials such as metals, oxides, liquids, and gases.

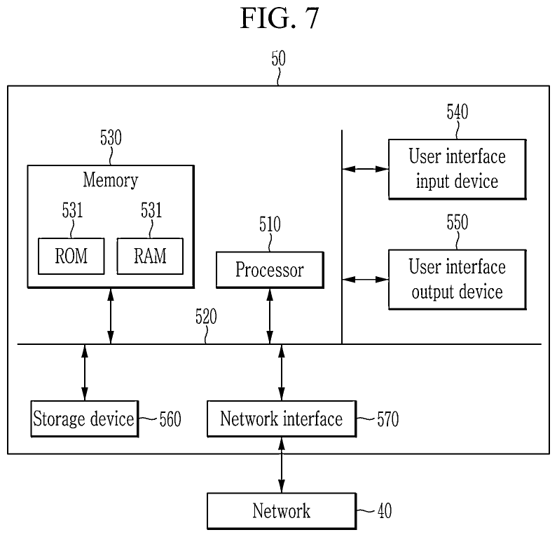

FIG. 7 is a drawing for explaining a computing device according to an embodiment.

Referring to FIG. 7, a chemical simulation apparatus and method according to an embodiment may be implemented by using a computing device 50.

The computing device 50 may include at least one of a processor 510, a memory 530, an user interface input device 540, an user interface output device 550 and a storage device 560 that communicate with each other through a bus 520. The computing device 50 may include a network interface 570 electrically connected to a network 40. The network interface 570 may send or receive signals to and from other entities through the network 40.

The processor 510 may be implemented in various types, such as a micro controller unit (MCU), an application processor (AP), a central processing unit (CPU), a graphic processing unit (GPU), a neural processing unit (NPU), and a quantum processing unit (QPU), may be any semiconductor device configured to execute instructions stored in the memory 530 or the storage device 560. The processor 510 may be configured to implement function and methods described above with reference to FIG. 1 to FIG. 6.

The memory 530 and the storage device 560 may include various types of volatile or non-volatile storage media. For example, the memory may include a read-only memory (ROM) 531 and a random access memory (RAM) 532. In the present embodiment, the memory 530 may be positioned in an interior or exterior of the processor 510, and the memory 530 may be connected to the processor 510 through various known means.

In some embodiments, at least some configuration or function of a chemical simulation apparatus and method according to embodiments may be implemented as a program or software executed by the computing device 50, and the program or software may be stored in a computer-readable medium.

In some embodiments, at least some configuration or function of a chemical simulation apparatus and method according to embodiments may be implemented by using hardware or circuitry of the computing device 50, or may also be implemented by using separate hardware or circuitry that may be electrically connected to the computing device 50.

According to embodiments described above, unlike a chemical simulation scale expansion method of the quantum computing-based quantum classical algorithm such as the conventional the VQE algorithm, physical properties and energy of a material and a molecular structure of which the scope of objects is expanded may be predicted by inputting energy of an object molecular structure calculated by the quantum classical algorithm as the training data of a machine learning-based the artificial neural network. Through the linkage of the quantum computing-based simulation with machine learning, a computational scale of an existing chemical simulation quantum classical algorithm may be increased by more than about 100 times. In addition, embodiments may be modified and optimized through appropriate selection of quantum simulation method, quantum classical algorithm, machine learning model, or the like depending on molecular structures to be simulated.

Particularly, since the scale of the chemical simulation is small (e.g., 10 to 20 qubit level) due to the limit of existing quantum computer hardware, it may be difficult to apply the quantum calculation to a computational chemistry problem of a practical scale. In addition, since a computational scale of the DMET, the FMO, or the like may not be expanded to a sufficiently large scale to have practicality, it is still difficult in the current situation to solve a chemistry problem in a practical scale. However, according to the present embodiment, the chemical simulation may be expanded to a sufficiently large scale by using a quantum computing result as of a learning input value a machine learning model, and linking a conventional quantum computing algorithm of a small scale with machine learning. In addition, since the quantum computing algorithm and the machine learning algorithm may be tuned in a direction desired by the user, each algorithm may be configured to become an optimal combination, and the quantum classical algorithm and the machine learning algorithm selected as such may complement calculation accuracy and calculation speed and scale of each other. While this disclosure has been described in connection with what is presently considered to be practical embodiments, it is to be understood that the disclosure is not limited to the disclosed embodiments, but, on the contrary, is intended to cover various modifications and equivalent arrangements included within the spirit and scope of the appended claims.

1. A chemical simulation apparatus, comprising:
a training data generation module configured to sample a molecular structure to be learned, to set a wave function, and to extract an energy value with respect to the molecular structure based on a quantum computing;
a fingerprint conversion module configured to convert structure calculated in the quantum computing into a fingerprint;
a learning module configured to perform a neural network learning by using the converted fingerprint as an input; and
a prediction module configured to predict material energy or physical properties by using the learned neural network.

2. The chemical simulation apparatus of claim 1, wherein the training data generation module is configured to set an ansatz comprising a quantum circuit configured to implement a quantum algorithm taking the sampled molecular structure as an input.

3. The chemical simulation apparatus of claim 2, wherein the training data generation module is configured to perform the quantum algorithm based on the wave function, and to optimize a ground state energy with respect to the molecular structure.

4. The chemical simulation apparatus of claim 1, wherein the training data generation module is configured to extract the energy value with respect to all molecular structures determined to be necessary for the neural network learning.

5. The chemical simulation apparatus of claim 1, wherein the fingerprint conversion module is configured to obtain the fingerprint by applying a symmetric function with respect to the molecular structure, and
wherein the fingerprint is generated as a fingerprint of a same structure with respect to a same molecular structure, regardless of translation and rotation.

6. The chemical simulation apparatus of claim 5, wherein the symmetric function comprises a symmetric function of a Gaussian type.

7. The chemical simulation apparatus of claim 1, wherein the learning module is configured to finish learning when a calculation error of a training set converges to a predetermined value or less.

8. The chemical simulation apparatus of claim 7, wherein the predetermined value is 10 mHa/atom, where mHa denotes milli-Hartree.

9. The chemical simulation apparatus of claim 7, wherein:
the learning module is configured to finish learning when a prediction accuracy of a test set not used for the neural network learning converges to the predetermined value, and to modify the training set when the prediction accuracy does not converge to the predetermined value;
the fingerprint conversion module is configured to convert the fingerprint with respect to the modified training set; and
the learning module is configured to perform the neural network learning by using the fingerprint converted with respect to the modified training set as an input.

10. The chemical simulation apparatus of claim 1, wherein the prediction module is configured to predict a new structure not comprised in the neural network learning.

11. A chemical simulation method, comprising:
sampling, by a computing device, a molecular structure to be learned;
setting, by the computing device, a wave function;
extracting an energy value with respect to the molecular structure based on a quantum computing;
converting a structure calculated in the quantum computing into a fingerprint;
performing a neural network learning by using the converted fingerprint as an input; and
predicting a material energy or physical properties by using the learned neural network.

12. The chemical simulation method of claim 11, further comprising setting an ansatz comprising a quantum circuit configured to implement a quantum algorithm taking the sampled molecular structure as an input.

13. The chemical simulation method of claim 12, further comprising performing the quantum algorithm based on the set wave function, and optimizing a ground state energy with respect to the molecular structure.

14. The chemical simulation method of claim 11, wherein extracting the energy value comprises extracting the energy value with respect to all molecular structures determined to be necessary for the neural network learning.

15. The chemical simulation method of claim 11, wherein converting into the fingerprint comprises obtaining the fingerprint by applying a symmetric function with respect to the molecular structure, and
wherein the fingerprint is generated as a fingerprint of a same structure with respect to a same molecular structure, regardless of translation and rotation.

16. The chemical simulation method of claim 15, wherein the symmetric function comprises a symmetric function of a Gaussian type.

17. The chemical simulation method of claim 11, wherein performing the neural network learning comprises finishing learning when a calculation error of a training set converges to a predetermined value or less.

18. The chemical simulation method of claim 17, wherein the predetermined value is 10 mHa/atom, where mHa denotes milli-Hartree.

19. The chemical simulation method of claim 17, wherein:
performing the neural network learning comprises finishing learning when prediction accuracy of a test set not used for the learning converges to the predetermined value, and modifying the training set when the prediction accuracy does not converge to the predetermined value;
converting into the fingerprint comprises converting the fingerprint with respect to the modified training set; and
performing the neural network learning comprises performing the neural network learning by using the fingerprint converted with respect to the modified training set as an input.

20. A computer-readable medium storing a program or instructions to enable a computer to perform a method, wherein the computer comprises a processor configured to execute the program or instructions, wherein the method comprises:
sampling a molecular structure to be learned;
setting a wave function;
extracting an energy value with respect to the molecular structure based on a quantum computing;
converting a structure calculated in the quantum computing into a fingerprint;
performing a neural network learning by using the converted fingerprint as an input; and
predicting a material energy or physical properties by using the learned neural network.
